(19) United States
(12) Patent Application Publication
(10) Pub. No.: US 2006/0134621 A1
(43) Pub. Date: Jun. 22, 2006

(54) Diagnostic test for attention deficit hyperactivity disorder

(76) Inventors: James M. Swanson, Irvine, CA (US); Robert K. Moyzis, Corona Del mar, CA (US)

Correspondence Address: CHRISTIE, PARKER & HALE, LLP, PO BOX 7068, PASADENA, CA, 91109-7068, US

(21) Appl. No.: 10/538,379

(22) PCT Filed: Dec. 15, 2003
(86) PCT No.: PCT/US03/40101

§ 371 Date: Nov. 22, 2005

Related U.S. Application Data
(60) Provisional application No. 60/433,045, filed on Dec. 13, 2002.

(51) Int. Cl. C12Q 1/68

(52) U.S. Cl. 435/6

(57) ABSTRACT

A two-pronged approach useful for diagnostic testing of ADHD is provided, comprising (1) a genetic test to determine the DRD4 genotype and (2) a test to characterize dopamine release to mental/physical/pharmacological probes.

### INTRODUCTION

ADHD is a diagnosis applied to children and adults who consistently display certain characteristic behaviors over a period of time. The most common behaviors fall into three categories: inattention, hyperactivity, and impulsivity.

To assess whether a person has ADHD, specialists consider several critical questions: Are these behaviors excessive, long-term, and pervasive? Are they a continuous problem, not just a response to a temporary situation? Do the behaviors occur in several settings or only in one specific place like the playground or the office? The person's pattern of behavior is compared against a set of criteria and characteristics of the disorder. These criteria appear in a diagnostic reference book called the DSM (short for the Diagnostic and Statistical Manual of Mental Disorders).

According to the diagnostic manual, there are three patterns of behavior that indicate ADHD. People with ADHD may show several signs of being consistently inattentive. They may have a pattern of being hyperactive and impulsive. Or they may show all three types of behavior.

According to the DSM, signs of inattention include:

becoming easily distracted by irrelevant sights and sounds

failing to pay attention to details and making careless mistakes

rarely following instructions carefully and completely

losing or forgetting things like toys, or pencils, books, and tools needed for a task

Some signs of hyperactivity and impulsivity are:

feeling restless, often fidgeting with hands or feet, or squirming

running, climbing, or leaving a seat, in situations where sitting or quiet behavior is expected

blurting out answers before hearing the whole question

having difficulty waiting in line or for a turn

Because everyone shows some of these behaviors at times, the DSM contains very specific guidelines for determining when they indicate ADHD. The behaviors must appear early in life, before age 7, and continue for at least 6 months. In children, they must be more frequent or severe than in others the same age. Above all, the behaviors must create a real handicap in at least two areas of a person's life, such as school, home, work, or social settings. So someone whose work or friendships are not impaired by these behaviors would not be diagnosed with ADHD. Nor would a child who seems overly active at school but functions well elsewhere.

Medications have been used to treat the symptoms of ADHD. Three medications in the class of drugs known as stimulants seem to be the most effective in both children and adults. These are methylphenidate (Ritalin), dextroamphetamine (Dexedrine or Dextrostat), and pemoline (Cylert). For many people, these medicines dramatically reduce their hyperactivity and improve their ability to focus, work, and learn. The medications may also improve physical coordination, such as handwriting and ability in sports. Recent research by NIMH suggests that these medicines may also help children with an accompanying conduct disorder to control their impulsive, destructive behaviors.

However, many parents are reticent to give their children such medications in the absence of a definitive, or as definitive as possible, diagnosis of ADHD. Thus, a diagnostic test that eliminates “false positives,” i.e., those individuals displaying many of the behavioral characteristics of ADHD but lacking the cognitive defects of the “true” disorder, while providing a more definitive diagnosis of those individuals manifesting both the behavioral symptoms and the cognitive defects, would be useful.

### BRIEF DESCRIPTION OF THE DRAWINGS

FIG. 1 Diagrammatic representation of the human DRD4 gene region. Exon positions are indicated by blocks (yellow: noncoding, orange: coding). The approximate positions of a 120 bp promoter region duplication (blue triangle), an exon 1 12 bp duplication (blue triangle), an exon 3 48bp VNTR (blue triangle), and two intron 3 SNPs are indicated. 2R through 11R variants of the 48 bp VNTR are indicated below exon 3 (blue), along with their worldwide population frequencies determined by PCR analysis (3,17).

FIG. 2 Nucleotide and amino acid sequences of VNTR motifs. The nucleotide and corresponding amino acid (red) sequences of 35 DRD4 exon 3 48 bp repeat motifs are shown. Prior nomenclature (2) for 19 of these motifs are indicated (α through ξ). The putative single step origin of most of these motifs is indicated, either as a recombination event (R) or a mutation event (M). For example, the seven motif is hypothesized to be a recombination between a 2 motif and a 3 motif (R 2/3) and the 8 motif is hypothesized to be a single point mutation of a 2 motif (M 2). Motifs 1 though 6, which account for the vast majority of observed haplotype variants (Table 1), are considered the progenitors. Motifs with no putative origin noted (for example, motif 15), have multiple possible progenitors.

FIG. 3 Proposed origin of DRD4 diversity. A simplified model for exon 3 48 bp repeat sequence diversity is shown, with only major recombination events indicated (FIG. 2). The major 2R, 4R, and 7R-alleles are shown in yellow, and the minor 3R, 5R, and 6R-alleles in gray, along with their hypothesized origins by unequal recombination (red arrows). Large red arrows indicate the putative multistep origin of the 7R-allele. Adjacent promoter region (L₁/S₁), exon 1 (L₂/S₂), and intron 3 (G-G/A-C) polymorphisms are indicated. The strong linkage of the L₁, L₂ and A-C polymorphisms with the DRD4 7R-allele is noted.

FIG. 4. Comparison among total work per body weight and peak heart rate in control subjects and subjects with ADHD. Both ADHD and control groups performed the same total work per body weight. Peak heart rate was higher in the control group but not significantly so.

FIG. 5. The effect of exercise on lactate levels. Both control subjects and subjects with ADHD had an increase in lactate level after exercise. Control subjects had a statistically significant higher lactate response to exercise compared with subjects with ADHD (*p<0.005).

FIG. 6. The effect of exercise on CA levels. Significant differences in EPI, NE, and DA responses to exercise were found between the control subjects and subjects with ADHD (*p<0.05).

### DETAILED DESCRIPTION OF THE INVENTION

Abbreviations

ADHD, attention-deficit/hyperactivity disorder

CA, catecholamine

NE, norepinephrine

EPI, epinephrine

DA, dopamine

HPA, hypothalamic-pituitary-adrenal

BMI, body mass index

VO2, oxygen consumption

CV, coefficients of variation

Overview.

Molecular genetic work on the Dopamine Receptor D4 (DRD4) suggests that the DRD4 7R “risk allele” may produce a “genocopy” of the disorder (ADHD), which is a partial syndrome with behavioral excesses but not cognitive deficits. The true syndrome may be the result of minimal brain damage (MBD), that is a result of fetal distress (hypoxia and hypotension) that may have selective effects on dopamine systems of the brain (in the striatal areas rich in dopamine terminals or the midbrain rich in dopamine cells).

A molecular genetic test using the DRD4 would identify potential “false positive” diagnoses, who have behavioral excesses but not cognitive deficits. Another test, based on brain imaging studies of adults suggests that the “true positive” ADHD individuals may have abnormal release of dopamine, and that this is the basis for a “dopamine deficit” that leads to cognitive as well as behavioral problems. We have also measured peripheral catechalomines (including dopamine) and shown a similar pattern in response to exercise (which itself should result in a release of dopamine as well as norepinephrine).

Accordingly, a two-pronged approach would be useful for diagnostic testing of ADHD: (1) a genetic test to determine the DRD4 genotype, to identify those children who have behavioral excesses (extraversion, creativity, high energy, etc.) that may prevent “false positive” diagnosis and (2) a blood/urine/saliva test to characterize dopamine release to mental/physical/pharmacological probes, to identify individuals with a neurochemical deficit.

Genetic Test

Associations have been reported of the 7-repeat (7R) allele of the human dopamine receptor D4 (DRD4) gene with both attention deficit/hyperactivity disorder (ADHD) and the personality trait of novelty seeking. This polymorphism occurs in a 48 bp tandem repeat (VNTR) in the coding region of DRD4, with the most common allele containing four repeats (4R), and rarer variants containing two to eleven. Here, we show by DNA resequencing/haplotyping of 600 DRD4 alleles, representing a worldwide population sample, that the origin of 2R- through 6R-alleles can be explained by simple one-step recombination/mutation events. In contrast, the 7R-allele is not simply related to the other common alleles, differing by greater than 6 recombinations/mutations. Strong linkage disequilibrium (LD) was found between the 7R-allele and surrounding DRD4 polymorphisms, suggesting this allele is at least 5-10 fold “younger” than the common 4R-allele. Based on an observed bias towards nonsynonymous amino acid changes, the unusual DNA sequence organization, and the strong LD surrounding the DRD4 7R-allele, we propose that this allele originated as a rare mutational event that nevertheless increased to high frequency in human populations by positive selection.

The DRD4 gene (1), located near the telomere of chromosome 11 p, is one of the most variable human genes known (2,3). Most of this diversity is the result of length and single nucleotide polymorphism (cSNP) variation in a 48 bp tandem repeat (VNTR) in exon 3, encoding the third intracellular loop of this dopamine receptor (2,3). Variant alleles containing two (2R) to eleven (11R) repeats are found, with the resulting proteins having 32 to 176 amino acids at this position. Interestingly, the frequency of these alleles varies widely. The 7R-allele, for example, has an extremely low incidence in Asian populations, yet a high frequency in the Americas (3).

A number of investigations have found associations between particular alleles of this highly variable gene and behavioral phenotypes (refs 4-8). While initial studies suggested that the 7R-allele of the DRD4 gene might be associated with the personality trait of novelty seeking (7,8), the most reproduced association is between the 7R-allele and attention deficit/hyperactivity disorder (ADHD) (refs 4-6,9). ADHD is the most prevalent disorder of early childhood, affecting an estimated 3% of elementary school children. As defined by DSM-IV criteria (10), ADHD consists of developmentally inappropriate inattention, impulsivity and hyperactivity with early onset (before the age of 7). Evidence of a strong genetic component of ADHD has come from a variety of twin, adoption, and family studies (11,12). The efficacy of methylphenidate in the treatment of ADHD indicated that genes in the dopamine pathway might play a role in the syndrome's etiology (9,13). Initial association studies found ADHD probands to exhibit an increased frequency of DRD4 7R-alleles in comparison to controls (4). Eight separate replications of this initial observation have now been reported (9). As in all association studies, however, one can not assume that the presence of a DRD4 7R-allele is either necessary or sufficient to “cause” ADHD. Further work will be required to understand the genetic/environmental factors underlying this behavior.

Nevertheless, given the likely functional importance of this change in the DRD4 protein, in a region that couples to G-proteins and mediates post-synaptic effects (14), these association studies have generated considerable interest (9). In particular, this association is consistent with the common variant-common disorder (CVCD) hypothesis, which proposes that the high frequency of many complex genetic diseases is related to common DNA variants (15,16). However, many questions remain as to the nature of the DRD4/ADHD association. One would like to know 1) if particular 7R-allele variants are associated with ADHD, 2) the population distribution of variant DRD4 alleles, and/or 3) whether the observed marker is in linkage disequilibrium (LD) with other etiologically relevant polymorphisms. Given the known high level of sequence polymorphism of this gene (2), PCR-based DNA resequencing is the most efficient and accurate method to address these questions.

Population Samples. Samples were obtained as reported previously (3,17). The origins of the 600 alleles reported in this study, based on geographical/ethnic origin, are as follows: North and South America, 12.7% (76 alleles), Europe, 36.7% (220 alleles), Asia, 27.3% (164 alleles), Africa, 20.3% (122 alleles), and Pacific, 3.0% (18 alleles). Lymphoblastoid cell lines have been established for most of these population samples, and methods for transformation, cell culture, and DNA purification described (3,17). For LD studies of the DRD4 4R-G-G SNP association, an additional 288 alleles (approximately equally derived from African, Asian and European sources) were used. All persons gave their informed consent prior to their inclusion in this study, carried out under protocols approved by the Human Subjects Committees at the participating institutions.

PCR Amplification and DNA sequencing. PCR amplification of the DRD4 promoter polymorphism was conducted as described (18,19). The program OLIGO 6.0 was used to select primer pairs for the exon 1 polymorphism (20) (5′-TGGGCCGCCGCATTCGT-3′ and 5′-GGTGGGTGTATCGCCGAGGGA-3′; 661-nucleotide product) and the exon 3 VNTR (2) (5′-CGTACTGTGCGGCCTCAACGA-3′ and 5′-GACACAGCGCCTGCGTGATGT-3′; 705 nucleotide product for the 4R-allele). For some amplifications of the VNTR, primers described previously were used (2). The alternative primers were chosen farther from the VNTR, to minimize out-of-register hybridization during amplification. PCR reactions were conducted in 25 microliter volumes, containing 100 ng genomic DNA, 200 micromolar dXTPs, 0.5 micromole of each primer, 1× PCR buffer (Qiagen), 1× Q-solution (Qiagen) and 0.625 units Taq DNA polymerase (Qiagen). Amplification was performed using Perkin-Elmer 9700 thermal cyclers. A 20 second, 96-degrees C. hot start was used, followed by 40 cycles of 95 degrees C. for 20 seconds and 68 degrees C. for 1 minute. Following a 4-minute chase at 72-degrees C., excess primers were eliminated with 0.5 units of Shrimp Alkaline Phosphatase (SAP, Amersham Life Science), 0.1 unit of Exonuclease I (Exo I, Amersham Life Science) and 1× SAP buffer (Amersham Life Science). The SAP/Exo I reaction was carried out at 37 degrees C. for 1 hour, followed by a 15-minute heat inactivation at 72-degrees C. The DNA from the SAP/Exo I reaction was used directly for DNA sequencing. For most individuals, the two allelic PCR products were first separated on 1.2-% agarose gels. DNA cycle sequencing was conducted by standard techniques, using ABI 377 and 3700 automated sequencers (21). DNA sequences of the DRD4 haplotypes reported in this paper have been submitted to GenBank (Accession numbers AF395210 through AF395264).

Kₐ/Kₛ and Allele age calculations. Kₐ/Kₛ ratios were calculated by standard methods (22,23). Putative recombinant haplotypes were not considered independent events. Allele age calculations were conducted by standard methods (refs 24-27). Briefly:

1) Calculated from Population Frequency.

E(t₁)=[−2p/(1−p)] ln (p), where E(t₁)=expected age, time is measured in units of 2N generations, and p=population frequency. For DRD4, p=19.2% for the 7R-allele and 65.1% for the 4R-allele. A generation time of 20-25 years and N=10,000 were assumed (regarded as a minimum estimate of the effective population size of modern humans during the period prior to recent growth; refs 24,26).

2) Calculated from Intra-Allelic Variation.

t=[1/ln(1−c)] ln [(x(t)−y)/(1−y)], where t=allele age, c=recombination rate, x(t)=frequency in generation t, and y=frequency on normal chromosomes. Assuming the origin of the 7R-allele was on a L₁L₂(7R)A-C haplotype, for the (7R)A-C association c=0.0000136 (from the average recombination rate per Mb times the VNTR-SNP distance), x(t)=97% (the percent of A-C SNPs associated with DRD4 7R-alleles), and y=13.9% (the percent of A-C SNPs associated with African DRD4 4R-alleles, assumed to be the “normal” allele). For the promoter polymorphism L₁(7R) association, c=0.000165, x(t)=90.8%, and y=61.9%.

Results and Discussion

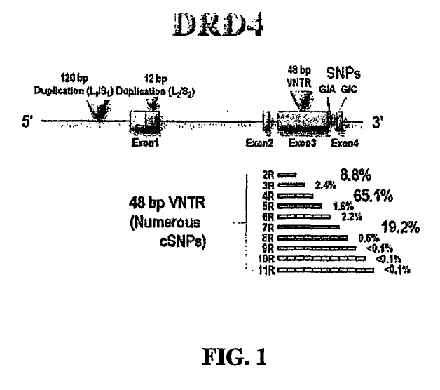

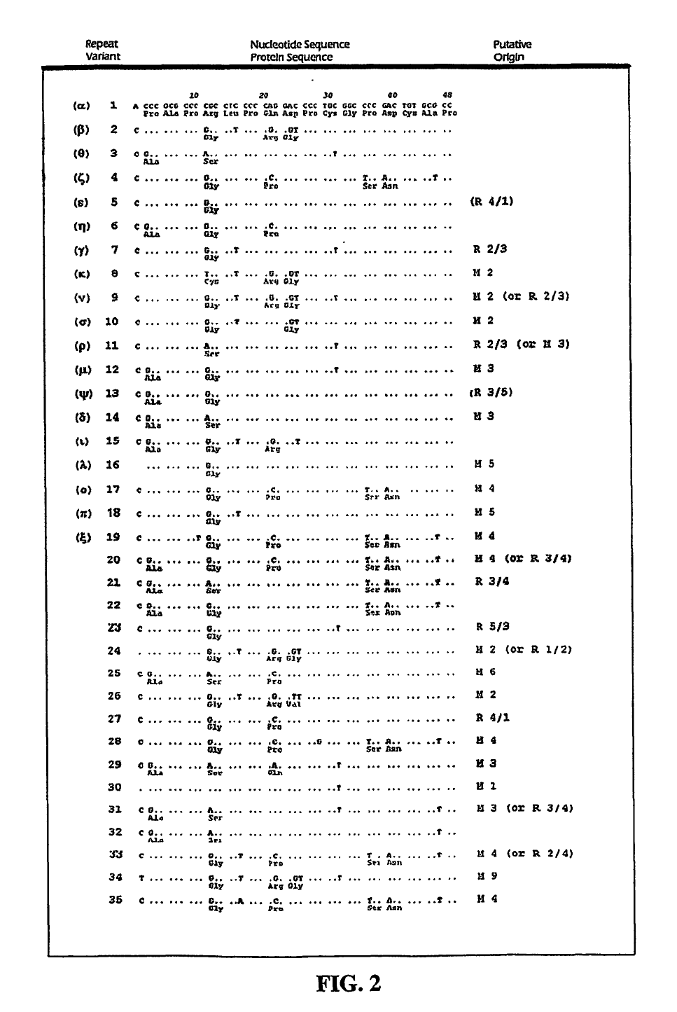

Primer sets were chosen to amplify the four exons of the highly GC-rich DRD4 gene (1), as well as the adjacent promoter region and splice junctions (FIG. 1). Initial resequencing of the entire promoter and coding region of the DRD4 gene from 20 ADHD probands (data not shown) uncovered a number of polymorphisms reported previously. These polymorphisms included two insertion/deletion polymorphisms, one in the promoter region (4.3 kb upstream of the VNTR; refs 18,19) and one in exon 1 (2.7 kb upstream of the VNTR; ref. 20; see FIG. 1). In addition, a number of new coding SNPs were uncovered in the exon 3 48 bp VNTR (2), as well as two previously unreported SNPs in intron 3, 20 nucleotides apart and approximately 350 bp downstream from the center of the VNTR (FIG. 1). Given the high level of VNTR polymorphism identified in this initial sample, a more extensive PCR-resequencing of 600 exon 3 VNTR alleles was conducted, obtained from a worldwide population sample (3,17) (Table 1 and FIG. 2). This sample contained individuals representing most major geographical origins (see Methods). The majority of individuals were heterozygotes, and the two allelic PCR products could be separated by gel electrophoresis prior to sequencing, providing unambiguous haplotypes. Altogether, we screened over 450,000 bp of genomic DNA and 2,968 48 bp repeats.

In the 600 chromosomes sequenced, 56 different haplotypes were found (Table 1). These haplotypes were composed of 35 distinct 48 bp variant motifs (FIG. 2), 19 of which were reported previously (ref 2; designated Alpha through Xi in FIG. 2). We propose that these DRD4 48 bp variant motifs are given numbers as shown, rather than the letters used previously (2), since there are not enough characters in the Greek alphabet. We propose that DRD4 exon 3 variants be designated in the format shown, i.e., the most common 4R allele being designated 4R(1-2-3-4), etc.

TABLE 1
Haplotypes of 600 ORD4 exon 3 alleles

| Allele | F | N | Haplotype |
|---|---|---|---|
| 2R | 0.033 | 55 | |
| | | 43 | 14 |
| | | 12 | 30-4* |
| 3R | 0.024 | 36 | |
| | | 16 | 1-7-4 |
| | | 9 | 1-2-4 |
| | | 4 | 1-1-33* |
| | | 3 | 1-9-4 |
| | | 1 | 1-2-22 |
| | | 1 | 1-2-21 |
| | | 1 | 1-2-31 |
| | | 1 | 1-2-32 |
| 4R | 0.651 | 250 | |
| | | 238 | 1-2-3-4 |
| | | 3 | 1-2-14-4 |
| | | 2 | 1-2-13-4 |
| | | 2 | 1-2-12-4 |
| | | 1 | 1-17-3-4 |
| | | 1 | 1-9-12-4 |
| | | 1 | 1-8-3-4 |
| | | 1 | 1-10-3-4 |
| | | 1 | 1-9-3-4 |
| 5R | 0.016 | 27 | |
| | | 12 | 1-3-2-3-4* |
| | | 4 | 1-2-13-34-4* |
| | | 3 | 1-2-2-3-4 |
| | | 2 | 1-2-6-5-4 |
| | | 2 | 1-11-2-3-4 |
| | | 1 | 1-3-2-14-4 |
| | | 1 | 1-2-6-23-4 |
| | | 1 | 1-2-3-9-4 |
| | | 1 | 1-2-3-27-4 |
| 6R | 0.022 | 24 | |
| | | 16 | 1-2-3-2-3-4 |
| | | 2 | 1-2-6-5-2-20 |
| | | 2 | 1-2-6-5-2-4 |
| | | 1 | 1-2-14-17-2-4 |
| | | 1 | 1-6-5-2-5-4 |
| | | 1 | 1-2-13-2-5-19 |
| | | 1 | 24-6-5-2-5-4 |
| 7R | 0.192 | 199 | |
| | | 177 | 1-2-6-5-2-5-4 |
| | | 5 | 1-2-6-5-2-5-19* |
| | | 3 | 1-2-6-5-2-3-4 |
| | | 3 | 1-2-6-5-13-5-4* |
| | | 2 | 1-8-25-5-2-5-4 |
| | | 2 | 1-2-3-5-2-5-4 |
| | | 1 | 1-2-6-5-2-13-4 |
| | | 1 | 1-2-29-17-2-5-4 |
| | | 1 | 1-2-6-2-2-5-4 |
| | | 1 | 1-8-25-5-2-3-4 |
| | | 1 | 1-2-6-16-2-3-4 |
| | | 1 | 1-2-6-5-2-14-4 |
| | | 1 | 1-2-3-17-2-5-4 |
| 8R | 0.006 | 6 | |
| | | 2 | 1-2-6-5-17-2-13-35* |
| | | 1 | 1-2-6-5-2-2-5-4 |
| | | 1 | 1-2-6-26-5-26-3-35 |
| | | 1 | 1-2-6-26-5-26-3-4 |
| | | 1 | 1-2-6-18-5-18-3-4 |
| 9R | <0.001 | 1 | 1-8-25-5-2-5-2-23-4 |
| 10R | <0.001 | 1 | 1-2-15-6-2-6-5-2-5-4 |
| 11R | <0.001 | 1 | 1-2-3-27-5-23-25-5-2-5-28 |

F, observed allele frequency in 2,836 chromosomes from 37 worlwide human populations (3, 17); [symbol] , allele number identified by sequence analysis in this study (non-R alleles were oversampled by 2-3-fold) haplotype are indicated using the repeat motif nomenclature prepared (FIG. 2).
Alleles with adjacent asterisks indicate common variants found only in a single population sample (2R [symbol] ; 3R1-11-33, Nas [symbol] ; ER 1-3-2-3-4, Chinese; 5R 1-2-13-34-4, Blakel; 78 1-2-6-5-19, Surul; 78 1-2-6-5-13-5-4, Nas [symbol] ; ER
1-2-6-5-17-2-13-35, [symbol] . Alleles with a single representation by definition were found in only one population.

We intentionally over sampled non-4R-alleles approximately two-fold, since little sequence variation was uncovered in the common 4R-allele (Table 1), even though it represents 65 percent of the world population frequency (3,17). Most of the haplotypes in this sample (85.7%) were found at frequencies less than 1% (Table 1). Looking at nucleotide diversity among variants defined by their VNTR number, the common 2R, 4R, and 7R-alleles exhibit the least diversity, with 78.2%, 95.2%, and 88.9% of the alleles respectively represented by the most common 2R(1-4), 4R(1-2-3-4), and 7R(1-2-6-5-2-5-4) haplotypes (Table 1). In contrast, while the 3R, 5R, 6R, and 8R alleles are rarer, they have proportionally more variants (Table 1). This unusual pattern of allele diversity is clearly not a simple length effect, i.e., longer alleles have greater diversity. Many population specific rare haplotypes were observed. Examples include the 2R(30-4) haplotype found only in the Surui (South America) sample, and the 5R(1-3-2-3-4) haplotype found only in the Han Chinese (Asian) sample (Table 1 and FIG. 2).

The pattern of nucleotide variation observed in the VNTR haplotypes is not random (FIG. 2). Most DNA sequence variants change the amino acid sequence, sometimes quite dramatically (i.e., Gln to Pro; FIG. 2). Although many of these variants are related mutational events (below), one can account for these relationships in calculating Kₐ/Kₛ (the ratio of the number of amino acid replacements per site divided by the estimate of the number of synonymous changes). Values of Kₐ/Kₛ greater than 1 are usually taken to be a stringent indicator of positive selection at the observed DNA segment (22,23). For a tandem repeat sequence, many assumed relationships can be inferred, and hence different Kₐ/Kₛ ratios calculated. For all assumed relationships of the DRD4 variants, however, Kₐ/Kₛ>1. For example, assuming that the most abundant 1 through 6-variant motifs (FIG. 2) all have a common origin, and that diversity was generated by both mutation and recombination (below), a Kₐ/Kₛ value of 3 is obtained. Expanding this analysis to include between-species divergence (a powerful method to improve these calculations) is not possible, due to the rapid de novo generation of variation in this VNTR in primate lineages (28).

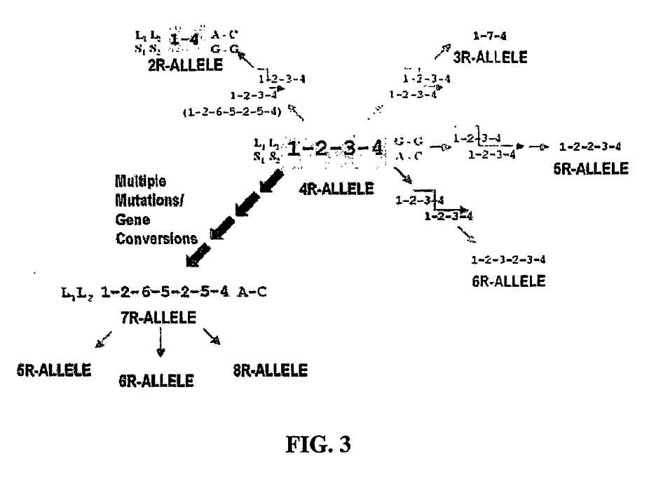

Standard approaches to defining evolutionary relationships between these haplotypes are not applicable, due to the repetitive nature of the DNA sequence (23). Based on the observed DNA sequences and their nucleotide variations, however, it is straightforward to propose a simple origin for the majority of these haplotypes (FIG. 3; Table 1). One-step recombination/mutation events between the most common alleles can account for nearly all of the observed variation of the 2R through 6R alleles. FIG. 3 is a simplified diagram of the most common recombination events proposed. While the inferred nucleotide sequence of an ancestral DRD4 cannot be determined, all alleles in a particular primate species appear to be derived from a relatively recent common ancestor (28). The most prevalent 4R-allele is proposed as the human progenitor allele, based on 1) limited sequence data reported for primate DRD4 4R-alleles (28), 2) the lower level of LD for polymorphisms surrounding this allele (as discussed below), and 3) the sequence motif arrangements of the non-4R alleles. Unequal recombination between two 4R(1-2-3-4) alleles would produce the observed common 2R though 6R alleles (FIG. 3). The position of crossover determines the resulting sequence. For example the most common 3R(1-7-4) and 3R(1-2-4) alleles differ only in the position of crossover, either within or after the second repeat (FIG. 3; Table 1). Thus, the known high frequency of unequal recombination between tandem repeats (29) can account for most of the observed diversity of the DRD4 gene.

In addition to unequal crossovers, single point mutations are evident in this population sample (Table 1 and FIG. 2). For example, with one exception, all 2R alleles worldwide have the sequence 2R(1-4) (Table 1). All twelve 2R alleles resequenced from Surui (South American) DNA were found to contain a single point mutation, the 2R(30-4) allele (Table 1 and FIG. 2). This mutation, a C to T change in the first repeat, does not alter the amino acid sequence, and likely has a recent (less than 10,000-20,000 year) origin (24).

In contrast, the formation of the observed 7R and higher alleles cannot be explained by simple one-step recombination/mutation events from the 4R(1-2-3-4) haplotype (FIG. 3). The generation of a 7R allele from the most prevalent 4R allele would require at least one recombination and 6 mutations to arise. Even allowing for more complicated gene conversion events, multiple low probability steps are needed to convert a 4R allele into a 7R allele (FIG. 3). For example, the central 5-variant motif found in the common 7R(1-2-6-5-2-5-4) haplotype could be produced by a recombination between two 4R-alleles. Recombination between the terminal 4-variant motif of one 4R-allele and the initial 1-variant motif of the second 4R-allele would yield a 7R(1-2-3-5-2-3-4) haplotype (FIG. 2). Three additional mutations of each of the two three-variant motifs in this putative 7R-haplotype are then required to produce the current 7R(1-2-6-5-2-5-4) haplotype. Four of these six nucleotide changes are nonsynonymous, altering the amino acid sequence (Ser to Gly, Gln to Pro, Ala to Pro, and Ser to Gly; FIG. 2). While gene conversion rather than mutation could be proposed as the mechanism to “insert” these nucleotide changes in a hypothetical 7R(1-2-3-5-2-3-4) allele, two unlikely events, one involving 7R-7R allele gene conversion, would be necessary (FIGS. 2 and 3).

None of these putative “intermediate” 7R haplotypes were observed in this worldwide population sample. Our sample included 47 7R-alleles sequenced from individuals of African origin, thought to contain populations with the greatest genetic diversity and age (24). It is unlikely, then, that “intermediate” 7R haplotypes exist at high frequency. It is not our intention, however, to propose a specific origin of the DRD4 7R-allele. Rather, we wish to emphasize that, based on DNA sequence analysis, the DRD4 7R-allele appears to be quite distinct from the common 2R through 6R alleles. It is impossible to determine if the origin of the DRD4 7R-allele was a single, highly unlikely event, or a series of unlikely events (FIG. 3).

Regardless of the mechanism of origin of the DRD4 7R-allele, it is clearly capable of participating in recombination events with the other alleles. Most of the rare 7R haplotypes observed appear to be recombination events, mostly with the common 4R(1-2-3-4) allele (Table 1). For example, the 7R(1-2-6-5-2-3-4) haplotype appears to be a recombination between a 4R(1-2-3-4) allele and a 7R(1-2-6-5-2-5-4) allele (Table 1 and FIG. 2). This origin was confirmed by analyzing SNPs outside the recombination region (see below). Further, the origin of some of the rare 5R and 6R alleles and all of the 8R and higher alleles can be explained by recombinations involving a 7R allele, since they contain the 6-variant motif, unique to the 7R allele (FIG. 2 and Table 1). Many of these 8R and higher alleles, however, appear to have more complicated origins, based on DNA sequence analysis (Table 1 and FIG. 2).

This model (FIG. 3) explains the apparent anomaly in the observed haplotype diversity noted above (Table 1), where the most abundant (and ancient, see below) 4R-allele has the lowest nucleotide diversity. If recombination is the predominant generator of diversity, then the majority of 4R/4R recombination events are predicted to have unchanged nucleotide sequence. Such events can only be inferred by recombination of outside markers. Only when out-of-register recombination occurs will new nucleotide sequence (and length) variants be generated (FIG. 3). The observed pattern of haplotype diversity is consistent with a predominantly “2-allele” system (4R and 7R), with most of the rarer variants generated by recombination from these two haplotypes (FIG. 3).

The unusual nature of the sequence organization of the DRD4 7R-allele, suggesting it arose as a rare mutational event, led us to determine if differences in LD exist between the 4R and 7R-alleles. The haplotype of two adjacent intronic SNPs (G/A-G/C; FIG. 1) could be directly determined, since they were present on the same PCR product used to amplify the 48 bp VNTR. Strong LD was found between the A-C SNP pair and the 7R-allele (FIG. 3). Ninety-seven percent of 7R-alleles were associated with the A-C SNP pair (66 out of 68 examined). The two 7R alleles associated with G-G SNPs were 7R/4R recombinant haplotypes, as determined originally from DNA sequence analysis (above). In contrast, both the G-G and A-C SNP pairs are associated with DRD4 4R-alleles (487 examined alleles). However, the G-G pair is most frequent, representing 86.1% of the African sample, but up to 98.6% of our Asian sample.

All African 7R-alleles were associated with the A-C haplotypes, while only 13.9% of African 4R-alleles were associated with the A-C haplotype. DNA sequence analysis of several chimp and bonobo samples (data not shown) indicates that the G-G SNP pair is likely the ancestral sequence (FIG. 3). Thus, it appears that the original DRD4 7R allele arose on this rarer A-C SNP background. A sample of 73 2R, 3R, 5R, and 6R-alleles showed approximately equal association with the G-G and A-C SNPs, consistent with their proposed recombinational origin from both the 4R and 7R-alleles (FIG. 3). Interestingly, all 26 Asian 2R-allele samples examined showed association with the A-C SNPs, suggesting their origin from recombinations involving 7R-alleles (FIG. 3).

Similar results were obtained for more distant promoter and exon 1 insertion/deletion polymorphisms (FIG. 1). In this case association was inferred indirectly from data obtained for our prior population studies (3,17) and PCR analysis of a subset of the individuals used in this study. For forty samples where parental DNA was also available and could be genotyped for these markers, phase could be directly inferred. Strong association was observed between the long (duplicated) L₁ promoter polymorphism (FIG. 1) and the 7R-allele (FIG. 3), with 90.8% of 7R-alleles associated with L₁ (607 alleles analyzed). In contrast, the L₁ polymorphism is coupled with only 61.9% of 4R-alleles (2102 alleles analyzed). While population specific variation was observed (for example, more L₁-4R coupling in Chinese than African populations), little overall L₁-4R linkage was detected (FIG. 3). The closer L₂ polymorphism in exon 1 (FIG. 1) was associated with 93.4% of 7R-alleles and 86.4% of 4R-alleles, a relative difference similar to that observed for the L₁-7R and L₁-4R association. The L₂/S₂ polymorphism is in a coding region, however, and selective constraints may be influencing allele frequency as well (30).

Standard methods of estimating coalescence time for these alleles are not applicable, given the repetitive nature of the region and the high recombination frequency. However, calculations of allele age based on the relatively high worldwide population frequency of the DRD4 4R and 7R-alleles suggest that these alleles are ancient (>300,000 years old) (refs 25,26; see Methods). On the other hand, calculations of allele age based on the observed intra-allelic variability (refs 26,27; see Methods) suggest the 7R-allele is 5-10 fold “younger” (30,000-50,000 years old). Such large discrepancies between allele ages calculated by these two methods are usually taken as evidence that selection has increased the frequency of the allele to higher levels than expected by random genetic drift (26). The absolute values of these estimates are greatly affected by the assumptions used in their computations, for example the assumed recombination frequency (26). We have used conservative estimates of recombination frequency, based on the average observed for the terminal 20 Mb of 11 p (31). Given the observed high recombination at this locus (Table 1 and FIG. 3), it is likely that the actual age of the 7R-allele is even younger, and further LD analysis will refine these estimates. The important conclusion, however, is that regardless of the parameters assumed, the relative age differences for the 4R and 7R-alleles calculated from intra-allelic variability remains large, while their population frequency suggests they are both ancient.

The simplest hypothesis to account for 1) the observed bias in nucleotide changes (Kₐ/Kₛ), 2) the unusual sequence organization of the DRD4 7R-allele, and 3) the strong LD surrounding this allele, is that the 7R-allele arose as a rare mutational event (or events), that nevertheless increased to high frequency by positive selection. Advantageous alleles usually take a long time to reach a frequency of 0.1, then increase rapidly to high frequencies (>0.9). While it is possible we are observing the recent expansion of a highly advantageous 7R-allele, it is more likely, we suggest, that this “two-allele” DRD4 system (FIG. 3) is an example of balanced selection. Such selection may be more pervasive in the human genome than generally thought (24). A balanced selection model proposes that both the 4R and 7R-alleles are maintained at high frequencies in human populations. A variety of mechanisms could be proposed for such balanced selection, ranging from heterozygote advantage to frequency-dependent selection (24). According to evolutionary game theory (32), the evolutionary payoff for a particular kind of personality will depend on the existing distribution of personality types. For example, high aggression may lead to high fitness if almost everyone is meek, but might result in low fitness when very common, as aggressive individuals would suffer the penalties of frequent conflict. This type of frequency-dependent selection might be expected to apply to many types of psychological variation, including those associated with this particular neurotransmitter receptor (refs 4-9).

Alternative explanations to the proposed positive selection, such as recent random bottlenecks, population expansion, and/or population admixture (24) are less likely to account for the observed results. Bottlenecks have certainly occurred during human migration and evolution (refs 33-35), and have undoubtedly influenced the current worldwide DRD4 allele frequency. Numerous population studies on other genes (refs. 24,33,35) have shown that an “Out of Africa” constriction of allele diversity (and an increase in LD) likely occurred. In the present study, a greater diversity (and lower LD) was found for African DRD4 4R-alleles in comparison to the remainder of our population sample, which is consistent with the “Out of Africa” hypothesis (24). While one could argue that the 7R-allele frequency was increased by chance during the Out-of-Africa expansion, this does not explain the unusual lack of diversity in African 7R-alleles. The most common L₁L₂-7R(1-2-6-5-2-5-4)-A-C haplotype (FIG. 3) is found at frequencies comparable to those found worldwide (>85%). It is difficult to imagine what type of bottleneck could produce such results, i.e., strong worldwide LD for a single allele (DRD4 7R) yet little LD for the remaining alleles. A model that is consistent with the observed results is the “weak Garden of Eden” (wGOE) hypothesis (24), where the DRD4 4R-allele would be hypothesized to be ancient and present in indigenous populations, while the 7R-allele was spread by the expansion out of (and into) Africa. In such a wGOE hypothesis, positive selection for the DRD4 7R-allele must still be proposed.

Although we suggest that a recent mutational origin and positive selection best account for the DRD4 7R-allele data, another possibility can not be ruled out. Given the highly unlikely recombination/mutation events required to generate the 7R-allele from the 4R-allele, a possibility worth considering is the importation of this allele from a closely related hominid lineage. What lineage that might be can only be speculated, but Neanderthal populations were present at the approximate time the 7R-allele originated. Under this model, the coalescence time for the 4R and 7R-alleles would then be ancient, with the importation occurring only recently, as measured by LD. Obviously, additional experimental work may clarify these speculations.

For the DRD4 locus, it is unlikely that selection for an adjacent gene can account for the proposed selection, given the distinct and unusual DNA sequence of the DRD4 7R-allele itself. If the DRD4 7R-allele originated roughly 40,000 years ago, one might ask what was occurring at that time in human history? It is tempting to speculate that the major expansion of humans that occurred at that time, the appearance of radical new technology (the upper Paleolithic) and/or the development of agriculture (24), could be related to the increase in DRD4 7R-allele frequency. Perhaps individuals with personality traits such as novelty seeking, perseverance, etc. drove the expansion (and partial replacement)? The speculation that migration could account for the current 7R-allele distribution has been proposed (34). In addition to such phenotypic selection, sexual selection could be operating as well. As originally defined by Darwin (36), “any advantage which certain individuals have over others of the same sex and species solely in respect of reproduction” will lead to increased offspring. If individuals with a DRD4 7R-allele have personality/cognitive traits that give them an advantage (multiple sexual partners, higher probability for mate selection, etc.) then the frequency of this allele will expand rapidly, depending on the cultural milieu. Perhaps cultural differences can account for some of the observed differences in DRD4 7R-allele frequency (3)? Obviously, determining the exact nature of the DRD4 selection, and its biochemical and behavioral basis, awaits further experimentation. Recent experiments, indicating that individuals with ADHD and possessing this unusual DRD4 7R-allele perform normally on critical neuropsychological tests of attention in comparison to other ADHD probands (6), point to but one of many areas of future investigation.

One may ask why an allele that appears to have undergone strong positive selection in human populations nevertheless is now disproportionately represented in individuals diagnosed with ADHD? The CVCD hypothesis (16) proposes that common genetic variation is related to common disease, either because the disease is a product of a new environment (so that genotypes associated with the disorder were not eliminated in the past) or the disorder has small effect on fitness (because it is late onset). For early onset disorders (such as autism, ADHD, etc.) we suggest entertaining the possibility that predisposing alleles are in fact under positive selection, and only result in deleterious effects when combined with other environmental/genetic factors. In this context, it is possible that prior selective constraints are no longer operating on this gene. It is also possible to speculate, however, that the very traits that may be selected for in individuals possessing a DRD4 7R-allele may predispose behaviors that are deemed inappropriate in the typical classroom setting, and hence diagnosed as ADHD.

### REFERENCES (GENETIC TEST)

1. Van Tol, H. H. M., Bunzow, J. R., Guan, H.-C., Sunahara, R. K., Seeman, P., Niznik, H. B. and Civelli, O. (1991) Nature 350,610-614.
2. Lichter, J. B., Barr, C. L., Kennedy, J. L., Van Tol, H. H. M., Kidd, K. K. and Livak, K. J. (1993) Human Molecular Genetics 2, 767-773.
3. Chang, F.-M., Kidd, J. R., Livak, K. J., Pakstis, A. J., and Kidd, K. K. (1996) Hum. Genet. 98, 91-101.
4. La Hoste, G. J., Swanson, J. M., Wigal, S. B., Glabe, C., Wigal, T., King, N., and Kennedy, J. L. (1996) Molecular Psychiatry 1, 21-24.
5. Swanson, J. M., Flodman, P, Kennedy, J, Spence, M. A., Moyzis, R, Schuck, S, Murias, M, Moriarity, J, Barr, C, Smith, M, et al., (2000) Neuroscience and Behavioral Reviews 24, 21-25.
6. Swanson, J. M., et al. (2000) Proc. Natl. Acad. Sci. USA 97,4754-4759.
7. Ebstein, R. P., Novick, O., Umansky, R., Priel, B., Osher, Y., Blaine, D., Bennett, E. R., Nemanov, L., Katz, M., and Belmaker, R. H. (1996) Nature Genetics 12, 78-80.
8. Benjamin, J., Li, L., Patterson, C., Greenberg, B. D., Murphy, D. L. and Hamer, D. H. (1996) Nature Genetics 12, 81-84.
9. Swanson, J., Deutsch, C., Cantwell, D., Posner, M., Kennedy, J., Barr, C., Moyzis, R., Schuck, S., Flodman, P., and Spence, M. A. (2001) Clinical Neuroscience Research 1, 207-216.
10. Am. Psychiatr. Assoc. (1994) DSM-IV: Diagnostic and Statistical Manual of Mental Disorder (Forth Edition) (Am. Psychiatr. Assoc., Washington, D.C.).
11. Faraone, S. V. and Biederman, J. (1994) Child Adolesc. Clin. North Am. 3, 285-291.
12. Thaper, A., Hohnes, J., Poulton, K., and Harrington, R. (1999) Br. J. Psychiatry 174, 105-111.
13. Volkow, N. D., Wang G. J., Fowler, J. S., Fischman, M., Foltin, R., Abumrad, N. N., Gatley, S. J., Logan, J, Wong, C, Gifford, A, et al., (1999) Life Sci. 65, 7-12.
14. Asghari, V., Sanyal, S., Buchwaldt, S., Paterson, A., Jovanovic, V., and Von Tol, H. H. M. (1995) J. Neurochem. 65, 1157-1165.
15. Collins, F. S., Guyer, M. S., and Chakravarti, A. (1997) Science 278, 1580-1581.
16. Zwick, M. E., Cutler, D. J., and Chakravarti, A. (2000) Ann. Rev. Genomics Hum. Genet. 1, 387-407.
17. Ding, Y.-C., Wooding, S., Harpending, H. C., Chi, H.-C., Li, H.-P., Fu, Y.-X., Pang, J.-F., Yao, Y.-G., Yu, J.-G. X., Moyzis, R., and Zhang, Y.-P. (2000) Proc. Natl. Acad. Sci. USA. 97, 14003-14006.
18. Seaman, M. I., Fisher, J. B., Chang, F.-M., and Kidd, K. D. (1999) Am. J. Med. Genet. 88, 705-709.
19. McCracken, J. T., Smalley, S. L., McGough, J. J., Crawford, L., Del'Homme, M., Cantor, R. M., Liu, A., and Nelson, S. F. (2000) Mol. Psychiatry 5, 531-536.
20. Catalano, M., Nobile, M., Novelli, E., Nothen, M. M., and Smeraldi, E. (1993) Biol. Psychiat. 34, 459-464.
21. Riethman, H. C., xiang, Z., Paul, S., Morse, E., Hu, X.-L., Flint, J., Chi, H.-C., Grady, D. L., and Moyzis, R. K. (2001) Nature 409, 948-951.
22. Kimura, M. (1968) Nature 217, 624-626.
23. Kreitman, M. (2000) Ann. Rev. Genomics Hum. Genet. 1, 539-559.
24. Harpending, H. and Rogers, A. (2000) Annu. Rev. Genoinics Hum. Genet. 1, 361-385.
25. Kimura, M. and Ohta, T. (1973) Genetics 75, 199-212.
26. Slatkin, M. and Rannala, B. (2000) Ann. Rev. Genomics Hum. Genet. 1, 225-249.
27. Serre, J. L., Simon-Bouy, B., Mornet, E., Jaume-Roig, B., Balassopoulou, A., Schwartz, M, Taillandier A, Boue J, Boue A., (1990) Hum. Genet. 84, 449-454.
28. Livak, K. J., Rogers, J., and Lichter, J. B. (1995) Proc. Natl. Acad. Sci. USA 92, 427-431.
29. Jeffreys, A. J., Neil, D. L., and Neumann, R. (1998) EMBO J. 17,4147-4157.
30. Seaman, M. I., Chang, F.-M., Deinard, A. S., Quinones, A. T., and Kidd, K. K. (2000) J. Exp. Zool. 288, 32-38.

31. International Human Genome Sequencing Consortium (2001) Nature 409, 860-921.
32. Smith, J. M. Evolution and the Theory of Games. (1982) Cambridge: Cambridge University Press.
33. Tishkoff, S. A., Dietzsch, E., Speed, W., Pakstis, A. J., Kidd, J. R., Cheung, K., Bonne-Tamir, B., Santachiara-Benerecetti, A. S., Moral, P., and Krings, M. (1996) Science 271, 1380-1387.
34. Chen, C., Burton, M., Greenberger, E., and Dmitrieva, J. (1999) Evol. Hum. Behav. 20, 309-324.
35. Reich, D. E., Cargill, M., Bolk, S., Ireland, J., Sabeti, P. C., Richter, D. J., Lavery, T., Kouyoumjian, R., Farhadian, S. F., Ward, R., and Lander, E. S. (2001) Nature 411, 199-204.
36. Darwin, C. The Descent of Man and Selection in Relation to Sex. (1874) New York: Merrill and Baker.
   Dopamine Test

A leading pathophysiologic hypothesis of attention-deficit/hyperactivity disorder (ADHD) is based on the notion of a catecholamine [CA; norepinephrine (NE), epinephrine (EPI), and dopamine (DA)] dysfunction (1, 2). This hypothesis suggests that the CA response to environmental stimuli is attenuated in ADHD and is derived primarily from observations that drugs such as methylphenidate and amphetamine—considered to be CA agonists—are effective in treating the symptoms of ADHD (1). Despite this compelling evidence, a definitive role of CA responsiveness in ADHD remains controversial (3).

Testing CA responsiveness in children with ADHD has proved to be complex. Protocols that elicit psychological stress using cognitive challenges—the bulk of research that has been done in children with ADHD—can yield measurable CA responses, but the stimulus is difficult to quantify or standardize (4, 5). Pharmacologic interventions that stimulate stress through, for example, rapid alterations in glycemia, are nonphysiologic, require extensive monitoring, and may not be acceptable or feasible for studies in children (6).

Here, we examined the possibility that exercise testing might be useful in differentiating CA responses to stress between subjects who have a diagnosis of ADHD and age- and gender-matched controls. Physical activity is widely known to be a powerful stimulus of the hypothalamic-pituitaryadrenal (HPA) and noradrenergic systems (7). Physical and mental stress each elicits physiologic responses that are mediated through the autonomic nervous system and endocrine system (8). In contrast to other types of stress-inducing protocols, exercise is a naturally occurring and physiologic stimulus of stress hormones, and the magnitude of the input (i.e. the work rate and duration) can be measured precisely and scaled to the capability of the subject. To our knowledge, testing the CA response using exercise has never been reported in children with ADHD.

We reasoned that the normal robust increase in circulating CA in response to exercise would be blunted in children with ADHD. To test this, we recruited treatment naïve children with newly diagnosed ADHD and measured CA response to an exercise test in which the work was scaled to each subject's physical capability.

The study was approved by the Institutional Review Board of the University of California, Irvine. Informed assent and consent were obtained from each subject and his or her parent or legally authorized representative, respectively, before the implementation of any study-related procedures. Standard, calibrated scales and stadiometers were used to determine height, weight, body mass index (BMI; wt/ht2), and BMI-for-age percentile (9).

Diagnosis of ADHD.

Families were recruited by a screening study for evaluation of children with ADHD at the University of California, Irvine, Child Development Center. Children between the ages of 7 and 12 y were eligible. For inclusion in the ADHD group, a diagnosis of ADHD-combined hyperactive/ impulsive subtypes was required. This was confined in a psychiatric interview of the parent about the child, by endorsement of at least six of the nine symptoms of inattention and six of the nine symptoms of hyperactivity/impulsivity on the Diagnostic Interview Schedule for Children. Children with a current history of depression, anxiety, epilepsy, or other medical conditions were excluded. All children who entered the study were naïve with respect to the use of stimulant medications to treat ADHD. Gender- and age-matched children who were healthy and had no history of ADHD were recruited as a control group.

Exercise Protocols.

We used an exercise that has been found to be effective in scaling the exercise input to the capabilities of healthy children as well as children with physiologic impairments (10). Each subject underwent two separate exercise testing sessions performed on different days within a week. First, we used a ramp-type progressive exercise test on an electronically braked cycle ergometer used extensively in children and adolescents (11). The second session consisted of a series of 10, 2-min bouts of constant-work rate cycle ergometry with 1-min resting intervals between each exercise bout. The work rate was individualized for each subject by finding the work rate corresponding to 50% of the difference between the anaerobic or lactate threshold of each subject (determined noninvasively from the ramp test) and the peak oxygen consumption (VO2) (12, 13). This approach was used, in contrast to the prolonged exercise testing, because although young children enjoy prolonged periods of physical activity, they find it hard to sustain constant exercise for more than several minutes at a time. In fact, typical bouts of exercise in children last only 25-30 s (14). The total duration of the second exercise protocol was 30 min (20 min of cycle ergometer exercise interspersed with 10 min of rest).

We calculated total external work performed by each subject [i.e. power×duration (kilojoules)] and normalized the total work performed to body mass. Finally, we measured the peak and end-exercise heart rate of each subject during the second exercise session.

Blood Sampling.

An indwelling venous catheter was inserted in the antecubital area. Blood samples were collected at pre-exercise (after 30 min of rest), during the last (tenth) 2-min exercise bout, 30 and 60 min after exercise.

EPI, NE, and DA.

EPI, NE, and DA were measured by a radioenzymatic technique based on the conversion of the CA to radiolabeled metanephrine and normetanephrine. This CA assay uses an extraction technique that eliminates substances that may inhibit the radioenzymatic assay. It also concentrates the CA to provide a more sensitive assay. One milliliter of plasma samples was extracted and then concentrated into a 0.1-mL volume before conversion into their radiolabeled metabolites. The assay has an extraction efficiency of 78%. The sensitivity of the assay is 10 and 6 pg/mL for NE and EPI. The intra-assay coefficients of variation (CV) are 4 and 13% for samples containing low levels of CA; variation is less for samples with high levels of CA. The inter-assay CV are 10% and 16%, respectively, for NE and EPI, so the assay is consistent over time. This technique is approximately 10 times more sensitive than the more commonly used assays and thus can reveal changes in venous CA levels that often go undetected (15).

Lactate.

Lactate was measured with the use of YSI lactate analyzer (YSI 1500, Yellow Springs, Ohio, U.S.A.). The intraassay CV was 2.8%, the interassay CV was 3.5%, and the sensitivity was 0.2 mg/dL.

Statistical Analysis.

Two-sample t tests were used to determine baseline differences in anthropometric variables, fitness variables, and circulating CA between control subjects and subjects with ADHD before the exercise protocol. Repeated measures ANOVA was used to test differences in response to the exercise bout between ADHD and control tests groups. For detecting possible differences in the pattern of response to exercise over time, the primary test of interest was the interaction of the between-subjects factor (group: ADHD versus control) and the within-subject factor (time: before, peak, 30 min after, and 60 min after). A post hoc single degree of freedom contrast to compare the baseline to peak change by group was tested to characterize whether the magnitude of response differed between the groups. Data are presented as mean±SEM.

Baseline Demographic Data

Ten newly diagnosed untreated male subjects (eight Caucasian, two Hispanic) with ADHD and eight healthy age-matched male controls (seven Caucasian, one Hispanic) volunteered for the study and met the screening criteria. Subject characteristics are presented in Table 2. No significant differences in age, height, weight, or BMI were found between control and ADHD groups.

TABLE 2
Anthropometric, peak O2, and peak work rate in subjects with ADHD and control subjects.

| | ADHD (a = 10) | | Control (n = 8) | |
|---|---|---|---|---|
| Age (years) | 8.4 ± 0.4 | (range 7-10) | 8.6 ± 0.5 | (range 7-11) |
| Height (cm) | 135 ± 3 | (range 117-145) | 138 ± 3 | (range 127-154) |
| Weight (kg) | 33.8 ± 3.8 | (range 22.3-52.7) | 34.4 ± 2.8 | (range 25.0-48.6) |
| [symbol] | 18.2 ± 1.1 | (range 14.4-25.1) | 17.8 ± 0.7 | (range 15.3-20.5 |
| Peak VO₂(L/min) [symbol] test | 1.01 ± 0.07 | (range 0.47-1.34) | 1.11 ± 0.09 | (range 0.83-1.48) |
| Peak VO₂(mL · mi [symbol] ⁻¹ · kg⁻¹) [symbol] test | 30.2 ± 1.7 | (range 21.2-37.2) | 32.8 ± 2.0 | (range 24.4-41.6 |
| lachlo thresthold (L/min) | 0.78 ± 8.86 | (range 0.48-1.03) | 0.79 ± 0.05 | (range 0.62-1.09) |
| Work rate for contant work rate protocol(mils) | 66.0 ± 4.7 | (range 45.0-85.0) | 64.0 ± 5.0 | (range 50.0-87.0) |
| Peak work rate (mils) [symbol] test | 85.7 ± 5.7 | (range 45-105) | 53.7 ± 7.1 | (range 80.0-130.0) |
| Heart rate during constant work rate protocol(beats/min) | 152.1 ± 4.6 | (range 124-172) | 159.9 ± 7.1 | (range 136-188) |

Data are presented as mean ± SEM

Effect of Brief Exercise
Peak VO2, Work Rate, and Heart Rate.

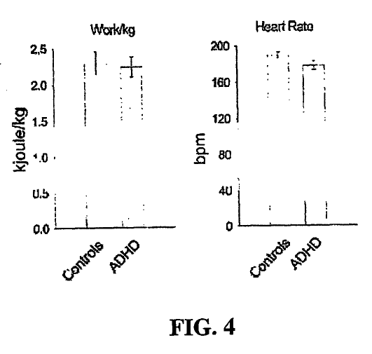

No significant differences were found in peak VO2, peak VO2 corrected for body weight, peak work rate, and lactate threshold between control subjects and subjects with ADHD (Table 2). The work rate performed per kilogram of body weight (kJ/kg) was almost identical in the ADHD and control groups. The control group reached a higher heart rate by end-exercise compared with the subjects with ADHD, but this difference was not significant (189.4±3.1 versus 178.1±5.1 respectively; FIG. 4).

Plasma Lactate and CA.

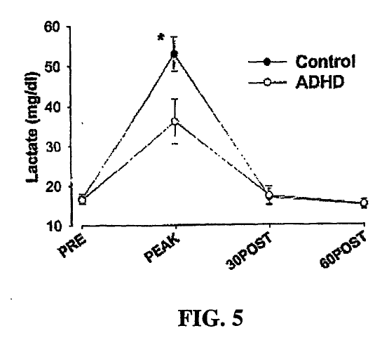

Plasma lactate increased significantly during exercise in both control and ADHD groups (p<0.001). There was a significant between-group difference in the lactate response to exercise, with a more prominent change in the control group (p<0.005; FIG. 5).

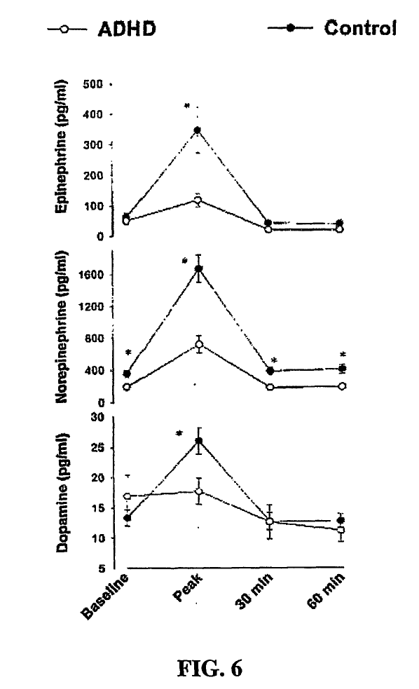

Baseline levels of NE and EPI were within the normal range for both children with ADHD and controls, suggesting that the blood drawing technique/timing was not stressful. Baseline plasma NE were significantly lower in the ADHD children (p<0.004). In response to exercise, mean NE levels rose in both groups; however, the rise in plasma NE was significantly greater in the control children compared with children with ADHD, reaching levels that were more than 2-fold higher in the control group (p<0.0005; FIG. 6).

No difference was found for baseline plasma EPI. EPI levels increased in both ADHD (p<0.002) and controls (p<0.006) after exercise. A statistically significant higher level of EPI at peak exercise was found in the control group (p<0.018). Baseline plasma levels of DA tended to be higher in the ADHD group, but this difference was not significant. After exercise, DA levels in the ADHD group did not change, whereas a significant increase was noted in the control group (p<0.016).

This study demonstrates for the first time abnormal responses of circulating EPI, NE, and DA accompanying cycle ergometer exercise in treatment-naive children with newly diagnosed ADHD. EPI and NE did increase in both control subjects and subjects with ADHD, but the responses were substantially blunted in the ADHD group even though the work performed did not differ from controls. Circulating DA increased significantly in the control subjects, but no increase was noted in the subjects with ADHD. Finally, a significant lower lactate response to exercise was found in ADHD, an observation consistent with a blunted CA response to exercise.

We selected a relatively high-intensity exercise protocol for this study because the CA response for work performed above the lactate threshold is known to be substantial (16). CA are increased with heavy exercise, in part because of CNS mechanisms. Activation of the HPA axis and sympathetic-adrenalmedullary activation leads to EPI release from the adrenal medulla and NE and, to a lesser degree, DA release, from nerve endings into the circulation (17, 18). Thus, exercise shares with other stresses (e.g. psychosocial) some common pathways that lead to increased CA output. In addition, the CA response to heavy exercise is further stimulated by systemic changes in acid-base balance and reduced oxygen availability to the working tissues (19).

Remarkably, the increase in circulating DA in response to exercise found in healthy children was absent in the subjects with ADHD (FIG. 6). Previous studies have demonstrated an increase in circulating DA in response to cycle ergometer (20) and resistance exercise (21) in adults, but this is the first documentation of the increase in circulating DA in healthy children. Whether the lack of a DA response in the periphery in ADHD is related, as some investigators propose, to a systemic “dopamine deficit” (22, 23) or, alternatively, simply to less stimulation of the adrenals in response to exercise has yet to be determined.

The present data provide indirect support for the connection between exercise and stimulation of HPA and noradrenergic systems in children with ADHD. This is in agreement with previous studies, for example, Hanna and et al. (24), Pliszka et al. (25), and Anderson et al. (4) who found substantially lower rates of EPI excretion in urine during cognitive testing in subjects with ADHD. These observations are consistent with earlier studies correlating academic performance and EPI excretion (26).

Most consistent with our observation of a blunted CA response to a physiologic stress in ADHD is the study of Girardi et al. (6). These investigators gave subjects an oral glucose load that led to an initial hyperglycemia followed by a rapid lowering of blood glucose and an accompanying CA burst. As in our study, Girardi et al. noted a blunted CA response in children who had a diagnosis of ADHD.

In studies that use exercise as an input to stimulate hormonal responses, a major potential confounding factor is whether the exercise input is comparable in the control and target groups. For example, if a protocol used a single work rate to compare hormonal response to exercise in two groups of subjects with high and low relative fitness, then the results might be confounded by the fact that the magnitude of the exercise input relative to the capability of each subject was different in the two groups (i.e. relatively higher in the unfit sample population). We achieved the goal of appropriately normalizing the work rates in the two populations by measuring fitness in each subject and adjusting the work rate input to the individual's capabilities. As seen in FIG. 4, we found no difference in the magnitude of the work rate input (either in absolute watts or when normalized to body mass) between the two groups. Thus, it is unlikely that the observed difference in the CA response was due to a lower relative work rate input in the ADHD group; rather, the data suggest abnormal CA regulation in the subjects with ADHD. It is noteworthy that the peak VO2 values observed in our study tended to be in the lower range of normal values that are typically reported for cycle ergometer exercise in children.

Although a strict matched-control design was not used, we sought the control group for this study from a general population and did not target children actively engaged in physical activity, as is often the case, for “normal” values in studies of exercise in children. It may well be and would not be surprising that in a large population comparison between healthy control children and children with ADHD that the latter would prove to have significantly lower levels of fitness.

A number of studies have been performed in normal subjects in which centrally acting pharmacologic agents were used to alter the CA response to exercise. Collomp et al. (27) and Stratton et al. (28) showed that benzodiaz-epines, which stimulate γ-aminobutyric acid receptors in the brain and blunt the CA response to a variety of stressors, markedly attenuated the EPI, NE, and DA response to exercise in the circulation. Interestingly, these investigators found that benzodiazepines attenuated the lactate response to exercise, similar to the observation we made in the children with ADHD (FIG. 5), and the blunted lactate response is likely explained by the reduced effect of peripheral CA on glucose metabolism.

Although the lactate levels in response to exercise were lower, the lactate threshold (the inflection point above which lactate concentrations in the circulation markedly increase) was not affected in the Stratton study. Similarly, we found no difference in the lactate threshold, determined noninvasively, between the subjects with ADHD and control subjects. These data showing that CNS suppression can lead to blunted peripheral CA responses supports the idea that there may exist a CNS dysregulation of CA in children with ADHD.

In addition to the exercise response, we found decreased baseline NE in the patients with ADHD. Surprising, little is known about circulating levels of CA in ADHD as the majority of the studies have focused on urine CA levels (2, 4). The lower NE that we found is consistent with some (29-31) but not all (32) previous studies. In the past, some investigators have questioned the relevance of peripheral (i.e. circulating) levels of CA in that they may not reflect CNS activity. However, it has become clear that activity of the peripheral nervous system does correlate with activity in the brain (33). Indeed, administering CA into the peripheral circulation (n.b., CA reportedly do not cross the blood-brain barrier) induce cognitive changes strongly suggestive of CNS effects (34).

Both physical exercise (35) and traditional pharmacologic treatment for ADHD with low doses of methylphenidate or amphetamine (36) increase executive function of the brain. Shepard et al. (37) demonstrated that long-term increases in physical activity were associated with improved academic performance in public school students. Despite the compelling physiologic role that exercise could play in the management of ADHD, to our knowledge, there have been no controlled studies designed to examine potential therapeutic benefits of exercise in ADHD. Our data pose some intriguing questions. First, does the blunted CA response to exercise also reflect a reduced exercise effect on executive function? Does repeated exercise (i.e. training) lead to an enhanced CA response to exercise or to other stresses in ADHD? Finally, does the CA response to exercise become normalized in the presence of traditional pharmacologic treatments with stimulant medications?

In summary, we found that the CA response to exercise was markedly reduced in children with ADHD. The agreement across studies examining the adrenomedullary and sympathetic responses in ADHD using various provocations, whether pharmacologic, cognitive, or physiologic, is remarkable. Our data suggest that CA excretion after a minimally invasive, nonpharmacologic exercise challenge in children with ADHD is deficient compared with healthy control children. These data are consistent with previous studies indicating that children with ADHD have lower CA responses to pharmacologic, physiologic, and cognitive challenges.

### REFERENCES (DOPAMINE TEST)

1. Zametkin A J, Rapoport J L 1987 Neurobiology of attention deficit disorder with hyperactivity: where have we come in 50 years? J Am Acad Child Adolesc Psychiatry 26:676-686.
2. Pliszka S R, McCracken J T, Maas J W 1996 Catecholamines in attention-deficit hyperactivity disorder: current perspectives. J Am Acad Child Adolesc Psychiatry 35:264-272.
3. Swanson J M, Sergeant J A, Taylor E, Sonuga-Barke E J, Jensen P S, Cantwell D P 1998 Attention-deficit hyperactivity disorder and hyperkinetic disorder. Lancet 351:429-433.
4. Anderson G M, Dover M A, Yang B P, Holahan J M, Shaywitz S E, Marchione K E, Hall L M, Fletcher J M, Shaywitz B A 2000 Adrenomedullary function during cognitive testing in attention-deficit/hyperactivity disorder. J Am Acad Child Adolesc Psychiatry 39:635-643.
5. King J A, Barkley R A, Barrett S 1998 Attention-deficit hyperactivity disorder and the stress response. Biol Psychiatry 44:72-74.
6. Girardi N L, Shaywitz S E, Shaywitz B A, Marchione K, Fleischman S J, Jones T W, Tamborlane W V 1995 Blunted catecholamine responses after glucose ingestion in children with attention deficit disorder. Pediatr Res 8:539-542.
7. Wittert G 2000 The effect of exercise on the hypothalamo-pituitary-adrenal axis. In: Warren M P, Constantini N W (eds) Sports Endocrinology. Humana Press, Totowa, pp 43-55.
8. Wasmund W L, Westerhohn E C, Watenpaugh D E, Wasmund S L, Smith M L 2002 Interactive effects of mental and physical stress on cardiovascular control. J Appl Physiol 92:1828-1834.
9. Kuczmarski R J, Ogden C L, Grummer-Strawn L M, Flegal K M, Guo S S, Wei R, Mei Z, Curtin L R, Roche A F, Johnson C L 2000 CDC growth charts: United States. Adv Data 314:1-27.
10. Tirakitsoontorn P, Nussbaum E, Moser C, Hill M, Cooper D M 2001 Fitness, acute exercise, and anabolic and catabolic mediators in cystic fibrosis. Am J Respir Crit Care Med 164:1432-1437.
11. Cooper D M, Weiler-Ravell D, Whipp B J, Wasserman K 1984 Aerobic parameters of exercise as a function of body size during growth in children. J Appl Physiol 56:628-634.
12. Armon Y, Cooper D M, Springer C, Barstow T J, Rahimizadeh H, Landaw E, Epstein S 1990 Oral 13C-bicarbonate measurement of CO2 stores and dynamics in children and adults. J Appl Physiol 69:1754-1760.
13. Barstow T J, Cooper D M, Epstein S, Wasserman K 1989 Changes in breath 13CO2/12CO2 consequent to exercise and hypoxia. J Appl Physiol 66:936-942.
14. Bailey R C, Olson J, Pepper S L, Barstow T J, Porszsasz J, Cooper D M 1995 The level and tempo of children's physical activities: an observational study. Med Sci Sports Exerc 27:1033-1041.
15. Kennedy B, Ziegler M G 1990 A more sensitive and specific radioenzymatic assay for catecholamines. Life Sci 47:2143-2153.
16. Urhausen A, Weiler B, Coen B, Kindermann W 1994 Plasma catecholamines during endurance exercise of different intensities as related to the individual anaerobic threshold. Eur J Appl Physiol Occup Physiol 69:16-20.
17. Pedersen B K, Hoffman-Goetz L 2000 Exercise and the immune system: regulation, integration, and adaptation. Physiol Rev 80:1055-1081.
18. Van Loon G R 1983 Plasma dopamine: regulation and significance. Fed Proc 42:3012-3018.
19. Schneider D A, McGuiggin M E, Kamimori G H 1992 A comparison of the blood lactate and plasma catecholamine thresholds in untrained male subjects. Int J Sports Med 13:562-566.
20. Nagao F, Suzui M, Takeda K, Yagita H, Okumura K 2000 Mobilization of NK cells by exercise: downmodulation of adhesion molecules on NK cells by catecholamines. Am J Physiol Regul Integr Comp Physiol 279:R1251-R1256.
21. Kraemer W J, Fleck S J, Maresh C M, Ratamess N A, Gordon S E, Goetz K L, Harman E A, Frykman P N, Volek J S, Mazzetti S A, Fry A C, Marchitelli L J, Patton J F 1999 Acute hormonal responses to a single bout of heavy resistance exercise in trained power lifters and untrained men. Can J Appl Physiol 24:524-537.
22. Levy F 1991 The dopamine theory of attention deficit hyperactivity disorder (ADHD). Aust N Z J Psychiatry 25:277-283.
23. Volkow N D, Wang G J, Fowler J S, Logan J, Franceschi D, Maynard L, Ding Y S, Gatley S J, Gifford A, Zhu W, Swanson J M 2002 Relationship between blockade of dopamine transporters by oral methylphenidate and the increases in extracellular dopamine: therapeutic implications. Synapse 43:181-187.
24. Hanna G L, Ornitz E M, Hariharan M 1996 Urinary catecholamine excretion and behavioral differences in ADHD and normal boys. J Child Adolesc Psychopharmacol 6:63-73.
25. Pliszka S R, Maas J W, Javors M A, Rogeness G A, Baker J 1994 Urinary catecholamines in attention-deficit hyperactivity disorder with and without comorbid anxiety. J Am Acad Child Adolesc Psychiatry 33:1165-1173.
26. Frankenhaeuser M 1971 Behavior and circulating catecholamines. Brain Res 31:241-262.
27. Collomp K, Fortier M, Cooper S, Long A, Ahmaidi S, Prefaut C, Wright F, Picot M, Cote M G 1994 Performance and metabolic effects of benzodiazepine during submaximal exercise. J Appl Physiol 77:828-833.
28. Stratton J R, Halter J B 1985 Effect of a benzodiazepine (alprazolam) on plasma epinephrine and norepinephrine levels during exercise stress. Am J Cardiol 56:136-139.
29. Shekim W O, Sinclair E, Glaser R, Horwitz E, Javaid J, Bylund D B 1987 Norepinephrine and dopamine metabolites and educational variables in boys with attention deficit disorder and hyperactivity. J Child Neurol 2:50-56.
30. Shekim W O, Javaid J, Dekirmenjian H, Chapel J L, Davis J M 1982 Effects of d-amphetamine on urinary metabolites of dopamine and norepinephrine in hyperactive boys. Am J Psychiatry 139:485-488.
31. Shekim W O, Dekirmenjian H, Chapel J L 1978 Urinary MHPG excretion in the hyperactive child syndrome and the effects of dextroamphetamine. Psychopharmacol Bull 14:42-44.
32. Brown G L, Ebert M H, Minichiello M D 1985 Biochemical and pharmacological aspects of attention deficit disorder. In: LM Bloomingdale (ed) Attention Deficit Disorder: Identification, Course, and Rationale. Spectrum Press Medical and Scientific Books, New York, pp 93-130.
33. Maas J W, Leckman J F 1983 Relationship between central nervous system noradrenergic function and plasma and urinary MHPG and other norepinephrine metabolites. In: Maas JW (ed) MHPG: Basic Mechanisms and Psychopathology. Academic Press, London, pp 33-44
34. van Zijderveld G A, van Doornen L J, van Faassen I, Orlebeke J F, van Dyck R, Tilders F J 1993 Adrenaline and the relationship between neurosomatism, aerobic fitness and mental task performance. Biol Psychol 36:157-181.
35. Tantillo M, Kesick C M, Hynd G W, Dishman R K 2002 The effects of exercise on children with attention-deficit hyperactivity disorder. Med Sci Sports Exerc 34:203-212.
36. Solanto M V 1998 Neuropsychopharmacological mechanisms of stimulant drug action in attention-deficit hyperactivity disorder: a review and integration. Behav Brain Res 94:127-152.
37. Shephard R J 1996 Habitual physical activity and academic performance. Nutr Rev 54:S32-S36.
   Two-Step Test

A two-pronged approach would be useful for diagnostic testing of ADHD: (1) a genetic test to determine the DRD4 genotype and (2) a blood/urine/saliva test to characterize dopamine release to mental/physical/pharmacological probes.

The genetic test is useful to identify those children who have behavioral excesses (extraversion, creativity, high energy, etc.) associated with identified DRD4 alleles that may result in a “false positive” diagnosis of ADHD; i.e., those children who exhibit the behavioral symptoms of ADHD but who do not have the cognitive defects associated with ADHD.

The dopamine test is useful to identify individuals with a neurochemical deficit affecting the dopamine systems of the brain, as is found in individuals having “true” ADHD. In this second prong, dopamine release is measured following an appropriate stimulus, as is known in the art. One such stimulus, described in detail herein, is exercise. However, other stimuli are known in the art and may be measured in a number of ways, such as bodily fluid tests, including blood, saliva and urine. Brain imaging studies may also be used; see, e.g., Volkow N D, Wang G J, Fowler J S, Logan J, Franceschi D, Maynard L, Ding Y S, Gatley S J, Gifford A, Zhu W, Swanson J M 2002 Relationship between blockade of dopamine transporters by oral methylphenidate and the increases in extracellular dopamine: therapeutic implications. Synapse 43:181-187.

All publications mentioned herein are incorporated herein by reference in their entireties. U.S. Provisional Application No. 60/433,045 is hereby incorporated by reference in its entirety. The publications discussed above, below and throughout the text are provided solely for their disclosure prior to the filing date of the present application. Nothing herein is to be construed as an admission that the inventor is not entitled to antedate such disclosure by virtue of prior invention.

While this invention has been described in detail with reference to a certain preferred embodiments, it should be appreciated that the present invention is not limited to those precise embodiments. Rather, in view of the present disclosure which describes the current best mode for practicing the invention, many modifications and variations would present themselves to those of skill in the art without departing from the scope and spirit of this invention. The scope of the invention is, therefore, indicated by the following claims rather than by the foregoing description. All changes, modifications, and variations coming within the meaning and range of equivalency of the claims are to be considered within their scope.

What is claimed is:

1. A method of testing a patient for attention deficit hyperactivity disorder (ADHD), comprising the steps of:
testing for the presence of a marker having a locus within a block of linkage disequilibrium surrounding the DRD4 7R allele in the patient, and
evaluating the level of dopamine release in the patient in response to a stimulus.

2. The method of claim 1, wherein the level of dopamine is evaluated using imaging of the brain.

3. The method of claim 1, wherein the stimulus is exercise.

4. The method of claim 1, wherein the level of dopamine release is measured in a body fluid of the patient.

5. The method of claim 4, wherein the body fluid is selected from the list consisting of blood, urine and saliva.

6. The method of claim 1, wherein the marker corresponds to the DRD4 7R allele.

7. A kit useful for diagnosing ADHD, comprising:
a polynucleotide corresponding to the DRD4 7R allele; and
reagents for evaluating dopamine levels in a bodily fluid.

8. A kit useful for diagnosing ADHD, comprising:
a polynucleotide corresponding to a marker the locus of which is within a block of linkage disequilibrium surrounding the DRD4 7R allele; and
reagents for evaluating dopamine levels in a bodily fluid.
